(19) United States
(12) Patent Application Publication
(10) Pub. No.: US 2010/0305506 A1
(43) Pub. Date: Dec. 2, 2010

(54) Method for Correcting Medical Fluid Injection Pressures Based on Usage

(76) Inventor: Theodore J. Fahrer, Dayton, OH (US)

Correspondence Address: Mallinckrodt Inc., 675 McDonnell Boulevard, HAZELWOOD, MO, 63042, US

(21) Appl. No.: 12/812,520

(22) PCT Filed: Jan. 15, 2009
(86) PCT No.: PCT/US09/31037

§ 371 Date: Jul. 12, 2010

Related U.S. Application Data
(60) Provisional application No. 61/022,003, filed on Jan. 18, 2008.

(51) Int. Cl. A61M 5/142

(52) U.S. Cl. 604/118

(57) ABSTRACT

Certain embodiments of the invention are directed to a medical fluid injector of the type that uses a motor to drive fluid from a fluid container by receiving electrical energy from a power source. The medical fluid injector has a motor drive control that delivers electrical energy to the motor. The motor drive control measures electrical energy delivered to the motor and computes a pressure value of the fluid delivered based upon the measured electrical energy. This pressure value is computed utilizing a calibrated relationship between the electrical energy delivered to the motor and resulting fluid pressure from the fluid delivered. The control further tracks a time factor over an operative life of the motor and alters the calibrated relationship based upon the time factor.

### RELATED APPLICATIONS

This application claims priority to U.S. provisional application Ser. No. 61/022,003, which was filed on 18 Jan. 2008, and which is entitled “Method for Correcting Medical Fluid Injection Pressures Based On Usage”.

### FIELD OF THE INVENTION

The present invention generally relates to injections of medical fluids, and more particularly, to methods for controlling injections of such medical fluids.

### BACKGROUND

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

In many medical environments, a medical fluid is injected into a patient during diagnosis or treatment. One example is the injection of contrast media into a patient to improve CT, Angiographic, Magnetic Resonance or Ultrasound imaging, using a catheter inserted into a patient's blood vessel.

One of the risks encountered when using contrast media in the aforementioned procedures is the risk of injecting the fluid at too high a pressure, and thereby stressing the patient's circulatory system, or damaging the tubes or other equipment that carries the contrast media to the patient. Another reason to have a maximum allowed delivery pressure is to alert the doctor of pressure increases caused by a restriction, such as would happen if the catheter delivering the contrast media was not in its proper position in a blood vessel. To detect and guard against too high of a delivery pressure, the physician or injector operator programs a maximum allowed pressure into the injector control. Based on this programmed value, the injector can sound an alarm, and/or shut-down.

It is desirable to measure such pressure. One method of doing this has been to depend on the measurement of the current draw of the motor powering the injector. This indirect method of measuring pressure is based on knowing the relationship between the electrical current to the motor, as well as the resulting output pressure of the injector. This relationship is established by calibrating each injector when it is new.

During the calibration process, the motor current required for a series of measured injection pressures is recorded. This recorded data is used to create a calibration curve for the specific injector. This calibration curve is used to convert a particular value of current to an associated value of pressure, or a particular value of pressure to an associated value of current. The curve may be recorded in many ways, such as in the form of a formula, a graph, or a table of numbers. The calibration curve is used by the injector control's logic.

A calibration curve is most accurate when the motor and the process it is driving remain unchanged from the moment at which the data for the calibration curve is recorded. If there are changes, a new calibration curve should be created to maintain maximum accuracy. However, performing another calibration is a time consuming and often expensive undertaking that often requires equipment not available at the medical facility at which the injector is used. Thus, field calibration can be difficult and is typically not performed.

The injector is a mechanical system, and mechanical systems are often subject to changes in the amount of friction from components such as motors, bearings, and seals. Potentially, the friction on these parts is initially greater than it is at the end of the break-in period. Although, after break-in, the friction remains relatively stable for a long period of time, late in the injector's life cycle, the friction may again increase (although a decrease is also possible) due to the beginning of mechanical failure, a lack of lubrication, and/or for other reasons.

The possible changes in the mechanical characteristics of an injector, as noted above, can alter the injector calibration. Since an injector is typically calibrated when it is nearly new, changes during the break-in period can cause the original calibration curve to quickly become inaccurate, and even if a re-calibration is performed after break-in, additional changes with age and wear will again cause the calibration to become inaccurate during the remaining life of the injector.

Accurate calibration would allow a physician's intended pressure limits to be more accurately monitored and responded to by the injector, making the injector more useful to the physician; however, the expense of field re-calibration to accomplish this objective, can be prohibitive.

### SUMMARY

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

In a first aspect, the invention features a medical fluid injector of the type that uses a motor to drive fluid from a fluid container by receiving electrical energy from a power source. The medical fluid injector has a motor drive control that delivers electrical energy to the motor. The motor drive control measures electrical energy delivered to the motor and computes a pressure value of the fluid delivered based upon the measured electrical energy. This pressure value is computed utilizing a calibrated relationship between the electrical energy delivered and fluid pressure. The motor drive control further tracks a time factor over an operative life of the motor and alters the calibrated relationship based upon the time factor.

Various refinements exist of the features noted in relation to the first aspect of the present invention. Further features may also be incorporated in the first aspect of the present invention as well. These refinements and additional features may exist individually or in any combination. The medical fluid injector may be of any appropriate size, shape, configuration, and/or type. For instance, the medical fluid injector may utilize one or more syringe plunger drivers of any appropriate size, shape, configuration, and/or type, where each such syringe plunger driver is capable of at least bi-directional movement (e.g., a movement in a first direction for discharging fluid; a movement in a second direction for accommodating a loading of fluid or so as return to a position for a subsequent fluid discharge operation), and where each such syringe plunger driver may interact with its corresponding syringe plunger in any appropriate manner (e.g., by mechanical contact; by an appropriate coupling (mechanical or otherwise)) so as to be able to advance the syringe plunger in at least one direction (e.g., to discharge fluid). Further, the medical fluid injector may be used for any appropriate application where the delivery of one or more fluids is desired, including without limitation any appropriate medical application (e.g., computed tomography or CT imaging; magnetic resonance imaging or MRI; single photon emission computed tomography or SPECT imaging; positron emission tomography or PET imaging; X-ray imaging; angiographic imaging; optical imaging; ultrasound imaging). The medical fluid injector may be used in conjunction with any component or combination of components, such as an appropriate imaging system (e.g., a CT scanner). For instance, information could be conveyed between the medical fluid injector and one or more other components (e.g., scan delay information, injection start signal, injection rate).

Any appropriate number of fluid containers (e.g., syringes and/or fluid-containing bags) may be integrated with the medical fluid injector in any appropriate manner (e.g., in the case of syringes, detachably front-loaded, rear-loaded, or side-loaded), any appropriate fluid may be discharged from a given fluid container of the medical fluid injector (e.g., contrast media, a radiopharmaceutical, saline, and any combination thereof), and any appropriate fluid may be discharged from a multiple container injector configuration in any appropriate manner (e.g., sequentially, simultaneously), or any combination thereof. In one embodiment, fluid discharged from a container by operation of the medical fluid injector is directed into a conduit, where this conduit is fluidly interconnected with the container in any appropriate manner and directs fluid to a desired location (e.g., into a patient). In the case that the fluid container is a syringe, the syringe may be characterized as having a syringe barrel and a plunger that is disposed within and movable relative to the syringe barrel. This plunger may interface with a syringe plunger drive assembly of the injector such that the syringe plunger drive assembly is able to advance the plunger in at least one direction, and possibly in two different, opposite directions.

As stated above, the motor drive control tracks a time factor over an operative life of the motor and alters the calibrated relationship based upon the time factor. In some embodiments, the time factor may include one or more of a measure of elapsed time since the injector or one or more particular components thereof were placed into service, a number of injections performed by the injector, and a cumulative duration of injections performed (e.g., at or exceeding a predetermined threshold pressure). For example, in some embodiments, the time factor may be a count of a number of injections performed by the injector (e.g., at or exceeding a predetermined threshold pressure). In other embodiments, the time factor is a measure of elapsed time since the injector or one or more particular components thereof were placed into service.

In some embodiments, the calibrated relationship that is utilized by the motor drive control to compute the pressure value may be altered to reflect an increase over time in fluid pressure for a given amount of electrical energy delivered. In some embodiments, the calibrated relationship may be altered to reflect a decrease, over time, in fluid pressure for a given amount of electrical energy delivered.

The motor of the medical fluid injector may be a rotary electrical motor. In such embodiments, the electrical energy delivered to the motor and measured by the motor drive control may be in the form of an electrical current, and the motor may produce an output torque in approximately proportional relation to the electrical current.

A second aspect of the invention features a method of adjusting a motor feedback current-verses-pressure relationship for a medical fluid injector. In this method, a sample of individual specimens of a medical fluid injector of the type noted above is studied, and a model of usage-related changes in efficiency of the medical fluid injector type is formed based upon parameters-of-usage. The motor drive control of a specific injection system is initially calibrated to compute of injection pressure from electrical energy delivered to the motor, but then the parameters-of-usage of that specific injector are monitored, and used to recompute the calibration of the motor drive control of the specific injector based upon the monitored parameters-of-usage.

Various refinements exist of the features noted in relation to the second aspect of the present invention. Further features may also be incorporated in the second aspect of the present invention as well. These refinements and additional features may exist individually or in any combination. For instance, the various features noted above in relation to the medical fluid injector of the first aspect may be utilized by the second aspect, individually or in any appropriate combination.

### BRIEF DESCRIPTION OF THE FIGURES

FIG. 1 is a perspective view of an exemplary medical fluid injector.

FIG. 2 is a perspective view of another exemplary medical fluid injector.

FIG. 3 is a flow chart of an exemplary method to deliver a volume of medical fluid without exceeding a maximum injection pressure.

FIG. 4 is a graph to demonstrate the relationship of old and new injectors, and drifts in calibration.

### DETAILED DESCRIPTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

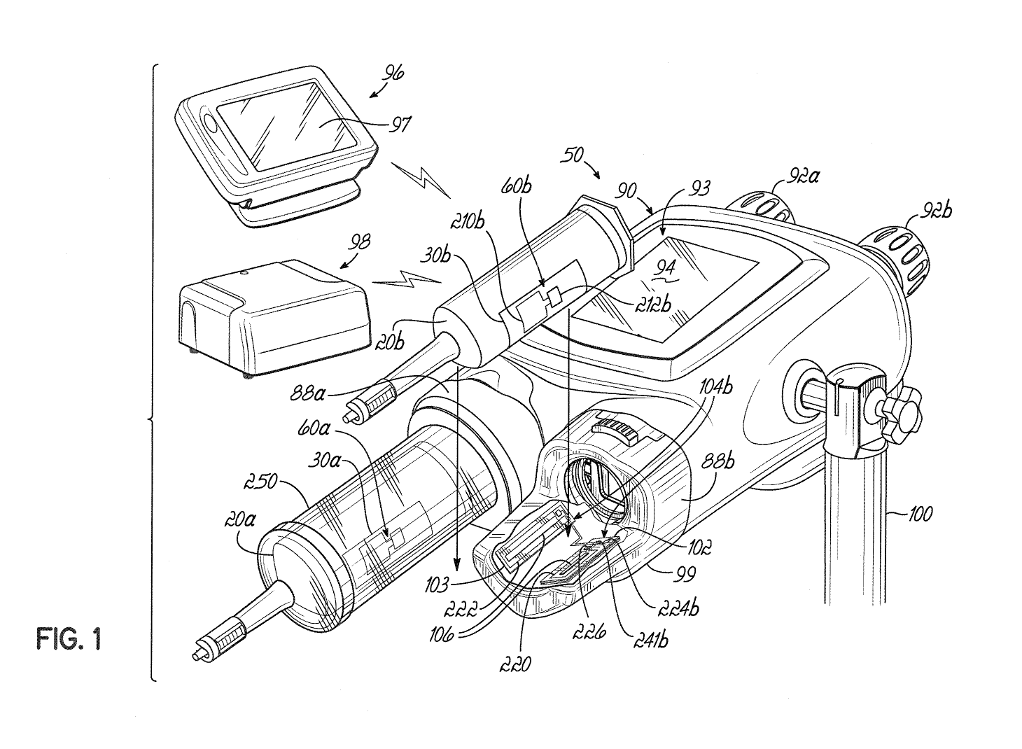

Referring to FIG. 1, a medical fluid injector 50 in accordance with the present invention may include various functional components, such as a powerhead 90, a console 96 and powerpack 98. Syringes 20a and 20b are mounted to the powerhead 90 in faceplates 88a and 88b of the powerhead 90, and the various injector controls are used to fill the syringe with medical fluid (e.g., contrast media for a CT, Angiographic or other procedure) that is subsequently injected into a patient under operator or pre-programmed control.

The powerhead 90 of the medical fluid injector 50 includes hand-operated knobs 92a and 92b for use in manually controlling movement of internal motors engaged to syringes 20a and 20b, and a touch screen display 94 for indicating to an operator the current status and operating parameters of the injector 50. The console 96 also includes a touch screen display 97 that may be used by an operator to remotely control operation of the powerhead 90. The console 96 (e.g., via the touch screen display 97) may be used to specify and store programs for automatic injection by the injector 50, which can later be automatically executed by the injector 50 upon initiation by an operator.

Powerhead 90 and console 96 connect through cabling (not shown) to the powerpack 98. Powerpack 98 includes a power supply for the injector, interface circuitry for communicating between the console 96 and powerhead 90, and further circuitry permitting connection of the injector 50 to remote units such as remote consoles, remote hand or foot control switches, or other original equipment manufacturer (OEM) remote control connections allowing, for example, the operation of the medical fluid injector to be synchronized with the x-ray exposure of an imaging system.

Powerhead 90 may be mounted to a stand, which includes a support arm 100 for supporting powerhead 90 for easy positioning of powerhead 90 in the vicinity of the examination subject. Console 96 and powerpack 98 may be placed on a table or mounted on an electronics rack in an examination room. Other installations are also contemplated however; for example, powerhead 90 may be supported by a ceiling, floor or wall mounted support arm.

The one or more syringes 20a, 20b are loaded into respectively one or both of the mounts or faceplates 88a, 88b that are attachable on powerhead 90 in a known manner. Exemplary details of the injector 50 are shown and described in U.S. Patent Application Publication No. 2006/0079765. In the present application, the injector 50 receives multiple syringes: a user-filled syringe having a volume of about 200 ml is mountable in a pressure jacket 250 of faceplate 88a, and a pre-filled syringe 20b having a volume in excess of about 90 ml or more may also be mountable in faceplate 88b. When the syringes are mounted, the plunger drives are operable to move plungers within the respective syringes 20a, 20b in a known manner. Exemplary operations of a powerhead 90 and injector control 93 are shown and described in U.S. Patent Application Publication No. 2006/0079842. Additional exemplary operations are described in U.S. Pat. Nos. 5,662,612, 5,681,286 and 6,780,170.

As noted, the powerhead 90 incorporates a touch screen 94, providing a user interface for displaying current status and setting operating parameters of the medical fluid injector 50, integrated into the surface of the housing of powerhead 90. Powerhead 90 may be mounted to a wheeled stand (partially shown at 100), which permits positioning of the powerhead 90 in the vicinity of the examination subject. The console 96 may be used by an operator to enter programs and control the operation of the powerhead 90 from a remote location in a known manner.

Elements of the injector control 93 may be incorporated into the powerhead 90 or may be incorporated in other elements of the injector such as the power supply 98 or console 96, or may be distributed among these elements. Further, the injector control 93 may be referred to herein or may include a motor drive control, which functions to control the drive motor of the injector. The powerhead touch screen 94 and console touch screen 97 display graphic representations of the respective syringes mounted in the powerhead 90, the operation of the powerhead, and the like.

Syringes 20a and 20b have RFID tags 60a and 60b thereon, integrated into labels 30a and 30b thereon. The RFID tags comprise a chip such as 212, and antenna loop such as 210b, which interact with radio frequency fields generated by antennae in the faceplate, as discussed below. The data contained on the RFID tag 60a or 60b may be displayed on the touch screens 94 and 97, allowing the operator to view and check the data at any time before as well as allowing data to be written into a history file on the RFID tag after an injection has been performed, which may be later accessed. Furthermore, the RFID tag 60a or 60b is capable of storing various data values; information regarding the use of the syringe and or its manufacture may be displayed on the touch screens 94 and 97. Further details on the use of RFID tags and the information that may be stored thereon is provided in PCT Application PCT/US2006/012620, filed Apr. 4, 2006, entitled SYSTEMS AND METHODS FOR MANAGING INFORMATION RELATING TO MEDICAL FLUIDS AND CONTAINERS THEREFOR. As noted therein, faceplate 88b has an outward extending cradle 99 that supports a first printed circuit (“PC”) board 102 and a second PC board 103, tuning circuit 226, and R/W RF driver circuit 224b and switching circuit 241b that collectively form an electromagnetic R/W device 1045. As noted in the above-referenced patent applications, that antennae 220 and 222 may be used in read-write operations in conjunction with an RFID tag 30b on syringe 20b. Furthermore, circuit boards 102 and 103 incorporate heaters 106 in the form of resistive traces on PC boards 102 and 104. The heaters 106 are electrically connected to the injector control via a cable or connector and are operable by the injector control circuitry to heat syringe 20b.

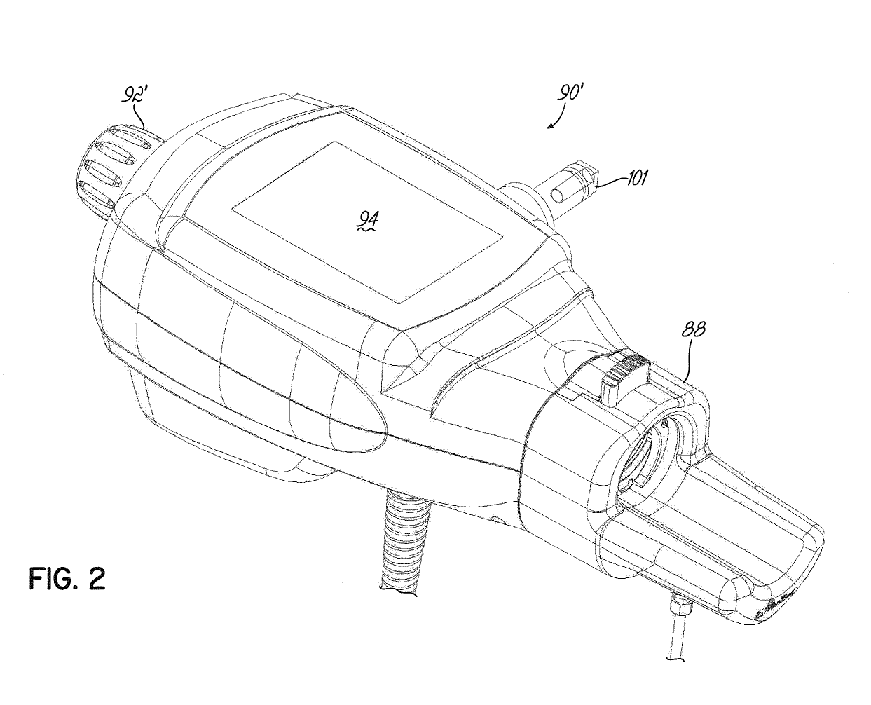

Although the powerhead 90 discussed above and shown in FIG. 1 is a dual head injector, embodiments of the present invention contemplate single head injectors as well. A suitable single-head powerhead 90′ is shown in FIG. 2. This single-head powerhead 90′ incorporates only a single faceplate 88, which may be of similar construction to the faceplate 88b shown and discussed above with reference to FIG. 1. A single knob 92′ is coupled to the drive mechanism within powerhead 90′ to allow manual operation of that drive system. Powerhead 90′ further incorporates a touch screen 94 for providing a user interface in the manner of the touch screen 94 shown in FIG. 1, although that user interface, by virtue of the single-head powerhead 90′, includes controls for a single syringe powerhead. Powerhead 90′ includes a mounting 101 for coupling to a mounting arm as is the case in dual-syringe powerhead 90 of FIG. 1, such as the arm 100 (see FIG. 1) of a stand.

As noted above, the control of the injector of FIG. 1 or FIG. 1a may be implemented by circuitry in any one of the powerhead 90, console 96 or power pack 98, or may be distributed among these elements. It will be appreciated that the control methods described below may be implemented in any medical fluid injector regardless of the specifics of its mechanical construction or the detailed layout or architecture of its digital or analog electrical components.

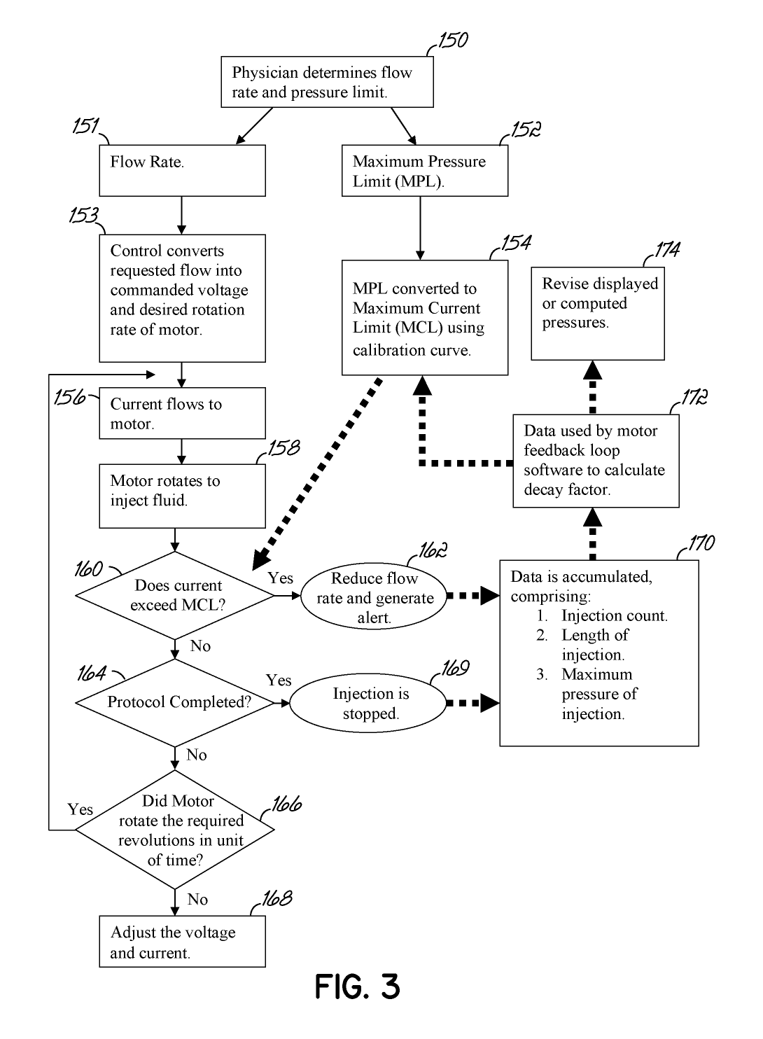

Referring to FIG. 3, the process used by a medical fluid injector in controlling an injection is described. Starting in block 150, an injector operator defines a desired injection protocol, including a requested rate of fluid flow, and a not-to-exceed maximum pressure limit. Generally, the pressure limit is a pressure that the medical fluid injector should not exceed during operation of the injection, regardless of the pressures that might be required to reach the requested flow rates. If the injector exceeds the set pressure limit, this is generally indicative of an error. The injector will reduce the flow rate and issue a warning to the operator so the injection has a chance to complete.

In block 151, the operator inputs the requested rate of fluid flow into the control system of the injector, and at block 152, the maximum pressure limit (abbreviated MPL for this discussion) is also input into the control. At block 153, the injector control (e.g., the motor drive control) converts the requested flow rate into a required rotational speed of the motor and, based upon calibration, computes a voltage to be applied to the motor that should produce the desired rotational speed. In block 154, the control, based upon calibration, converts the MPL into an equivalent MCL (maximum current limit). This MCL is available to be used at block 160, as indicated by the bold dotted arrow.

At block 156, the voltage computed in block 153 is applied to the motor, and current is delivered. This causes the motor to rotate to inject fluid as shown in block 158. In block 160, the control (e.g., the motor drive control) evaluates the current being delivered to the motor to determine whether the current exceeds the MCL computed in block 154. If it does, an alarm is annunciated, and the flow rate is reduced at block 162. If the current does not exceed the limit, then at block 164, the control system determines whether the protocol has been completed (e.g., if the desired fluid volume has been delivered). The first time that block 164 is visited, typically the injection will have just begun, and control will pass to block 166.

In block 166, the control determines whether the desired motor speed has been achieved (e.g., whether, during a unit time, the required number of motor rotations to deliver the requested rate of flow into the patient, as set in block 151, has occurred). If the motor is rotating at the rate required, control returns to block 156, and the injection proceeds. If the rotation rate is not in accordance with the protocol, then at block 168, the control (e.g., the motor drive control) adjusts the applied voltage on the motor, thus also adjusting the current applied and the flow rate. In the case where the flow rate is not sufficient, the voltage and current increase, to a greater level at block 168, and in the case where the flow rate is too great, the voltage and current decrease. After the adjustment, control returns to block 156, and the injection proceeds.

Ultimately, in a pass through the loop, the desired volume will have been delivered, and control will pass from block 164 to block 169, and the injection will halt.

After an injection, or after each of a set of injections, or on a period basis such as daily, monthly or weekly, additional calibration steps are performed as illustrated in blocks 170 and 172 in FIG. 3, which will be elaborated upon below.

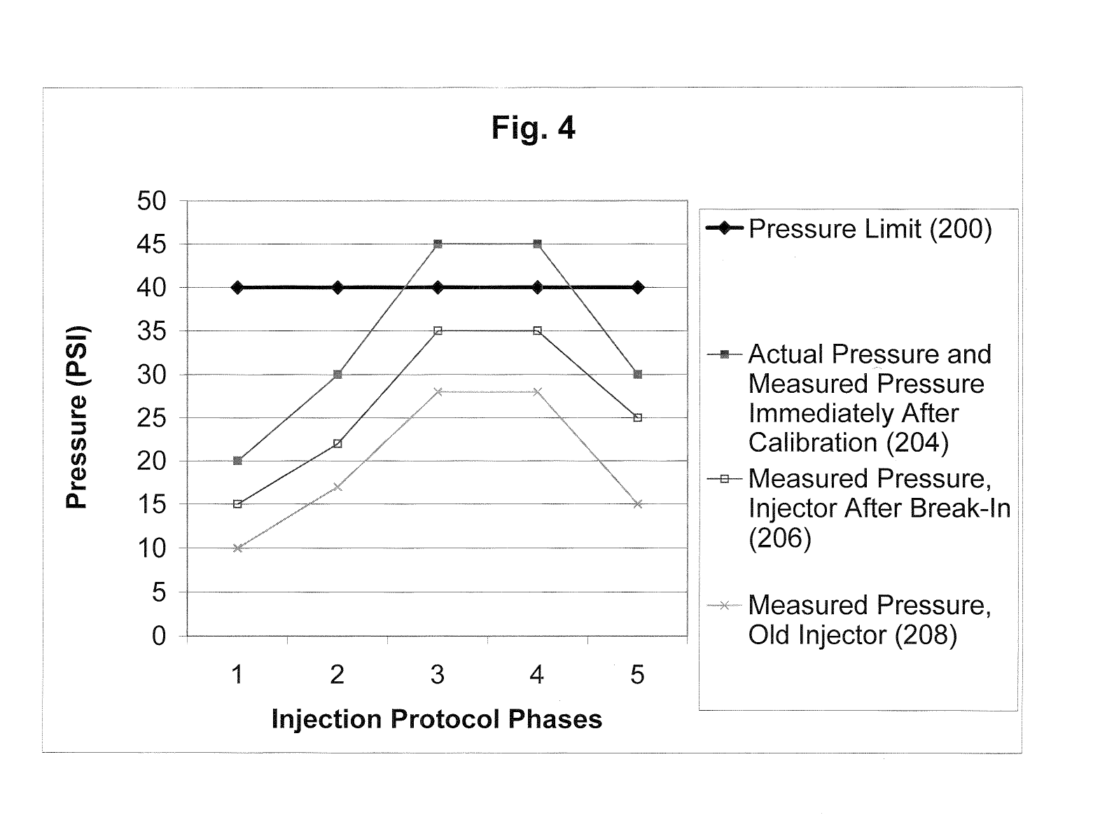

FIG. 4 illustrates the problem addressed by the recalibration steps 170 and 172 in FIG. 3. FIG. 4 illustrates, for a flow rate profile to be generated by an injector, a pressure limit 200, and three pressure profiles 204, 206 and 208, with symbols provided for ease of visual identification. The numbers along the Y (vertical) axis and the X (horizontal) axis are arbitrary, and are present only for purposes of providing the exemplary description below.

FIG. 4 illustrates the pressures generated to accomplish an injection protocol having five distinct phases of injection, each phase having a different flow rate. Phase 1 is at a relatively low flow rate, phase 2 is at a relatively low flow rate that is slightly larger than the flow rate of phase 1, phases 3 and 4 are at a relatively higher flow rate, and phase 5 returns to a relatively low flow rate.

Line 200 represents a maximum pressure limit for the illustrated injection protocol. As noted above, typically the pressure limit, along with a flow rate(s) and injected quantity(ies), is input by the operator to the control system of an injector to define its operation. In the illustrated example, the pressure limit is a constant maximum pressure limit of forty PSI, held constant throughout the requested injection protocol. In another example, the pressure limit could vary over time, or be computed for each requested flow rate of the injection protocol, in which case the line 200 angle upwards and/or downwards at different data points (times) during the procedure, so the line would not be horizontal.

Line 204 (square shapes) represents the actual pressure profile generated by an injector in achieving the requested flow rates for the protocol. In the illustrated example, there is a fault in the line or another source of overpressure that causes the applied pressure to increase above the pressure limit during the third and fourth phases of the injection, a condition that should trigger an error signal or halt the injection, as appropriate.

If it is assumed that the injection is performed with a new or refurbished injector having a new motor and drive train, which has not changed in mechanical behavior significantly since the calibration process was performed, the pressure measured by the injector control will be closely approximate to the actual pressure applied. In this case, the measured pressure will closely follow the line 204, and the injector control will detect the overpressure condition in step 160 of FIG. 3.

However, a different result may occur in the event that the mechanical behavior of the injector changes (e.g., significantly) over time. For example, the measured pressure may change after break-in of the injector. Specifically, as noted above, break-in of the injector may reduce the friction of the injector drive and/or increase the efficiency of the motor, in which case, the electrical drive (e.g., motor current) required to generate the requested flow rate(s) for the exemplary protocol will be reduced after break-in. If, as may be typical, the injector control (e.g., the motor drive control) is not recalibrated after break-in, the control will compute a measured pressure that is lower than the actual pressure being applied, due to calibration error.

Line 206 represents the pressure measured by the injector control performing the same procedure as line 204, after injector break-in. As can be seen, although the actual pressure applied has not changed, the measured pressure has been reduced due to the reduction in applied current caused by greater efficiency of the injector drive train. In the extreme case illustrated, the measured pressure does not exceed the pressure limit, even though the actual pressure being delivered has exceeded the pressure limit. Thus, an injection protocol that should have generated an error based on overpressure, fails to do so due to calibration error.

The deviation of measured pressure from actual pressure may become more severe over time. For example, an old injector may become more efficient over its lifetime, as further break-in of its component parts, and in particular, the electrical motor, cause the injector to become more efficient.

Line 208 represents the pressure measured by the injector control performing the same procedure, on an old injector that has become more efficient in the manner described above. Here again, the pressures that the injectors control system calculates for the old injector, are based on the calibration when the injector was new and frictional losses were high. Line 208 is even further below the pressure limit line 200, thus presenting even more opportunity for missing an overpressure condition.

It will also be appreciated that injection pressure, as computed from motor current, tracked and retained as part of a procedure summary. Due to the calibration changes described above, these pressure figures may show a number less than the actual pressure achieved.

It will be appreciated that injector wear may have an opposite effect than that illustrated in FIG. 4. That is, a given injector may become less efficient with break-in or with age. Or, there may be interplay of countervailing factors; initially, the injector may be come more efficient and then become less efficient, over its useful life, or vice-versa.

Referring again to FIG. 3, in an embodiment of the present invention, blocks 170 and 172 are included in the motor feedback process. These blocks compute a decay factor for friction and efficiency changes that may be experienced by an injector over its useful life. Block 170 illustrates exemplary data that can be collected by the injector during its operation, to keep track of the usage of the injector and the motor thereof, so that its usage progression through the break-in period and beyond is known. Data collected can include, for example, the number of injections performed by the injector, the length of such injections, and the pressure at which the injections were carried out. In block 172, the motor usage data can be used to compute a decay factor. The decay factor may, for example, model the typical percentage increase or decrease in overall injector efficiency over time or injection count, with additional modeling parameters for injection length and or injection pressures experienced. The model may be developed through empirical data from life testing of injectors of the same type, and included within the control software. In block 172, the friction decay model is used to modify the calibration of the injector, and thus correct computation of MCL in step 154, so that the motor current is compared to an MCL value that has been calibrated for the current stage of life of the medical fluid injector. Similarly, at step 174, all displayed or computed pressures derived by the injector from motor current, are computed using a revised calibration based upon the decay factors computed in via steps 170 and 172.

By improving the calibration of the control software, that control software can more accurately reflect achieved injection pressures, and more accurately compute the MCL used to detect overpressure conditions, potentially avoiding or forestalling physical recalibration of the injector that might otherwise be needed to achieve a similar level of accuracy.

While the various principles of the invention have been illustrated by way of describing various exemplary embodiments, and while such embodiments have been described in considerable detail, there is no intention to restrict, or in any way limit, the scope of the appended claims to such detail. Additional advantages and modifications will readily appear to those skilled in the art.

For example, in the described embodiments, systems of the described embodiments relate to containers of medical fluids. Examples described in detail relate to contrast media and syringes containing that media. In alternative embodiments, the medical fluid container may be a bag filled with a medical fluid, which is compressed to inject medical fluid, using a mechanical system that is subject to the same calibration as the systems described above.

There are many known structures for mounting a syringe to a power injector, and those shown and described herein are only two such structures. The inventions claimed herein can be applied to any appropriate injectors having any type of structure(s) for mounting a syringe or other fluid containers thereto.

When introducing elements of the present invention or various embodiments thereof, the articles “a”, “an”, “the”, and “said” are intended to mean that there are one or more of the elements. The terms “comprising”, “including”, and “having” are intended to be inclusive and mean that there may be additional elements other than the listed elements. Moreover, the use of “top” and “bottom”, “front” and “rear”, “above” and “below” and variations of these and other terms of orientation is made for convenience, but does not require any particular orientation of the components.

As various changes could be made in the above-described aspects and exemplary embodiments without departing from the scope of the invention, it is intended that all matter contained in the above description shall be interpreted as illustrative and not in a limiting sense.

What is claimed is:

1. A medical fluid injector, comprising:
a fluid container;
a motor for driving fluid from the fluid container; and
a motor drive control that:
delivers electrical energy to the motor;
measures the electrical energy delivered to the motor;
computes a pressure value of the fluid delivered from the fluid container based upon the electrical energy that is measured, wherein the pressure value is computed utilizing a calibrated relationship between the electrical energy delivered and fluid pressure;
tracks a time factor over an operative life of the motor; and
alters the calibrated relationship based upon the time factor.

2. The injector of claim 1, wherein the time factor is a count of a number of injections performed by the injector.

3. The injector of claim 1, wherein the time factor is a measure of elapsed time since the injector or one or more particular components thereof were placed into service.

4. The injector of claim 1, wherein the calibrated relationship is altered to reflect an increase, over time, in fluid pressure for a given amount of electrical energy delivered.

5. The injector of claim 1, wherein the calibrated relationship is altered to reflect a decrease, over time, in fluid pressure for a given amount of electrical energy delivered.

6. The injector of claim 1, wherein the time factor comprises one or more of:
a measure of elapsed time since the injector or one or more particular components thereof were placed into service;
a count of a number of injections performed by the injector; and
a cumulative duration of injections performed.

7. A method of adjusting a motor feedback current-verses-pressure relationship for a medical fluid injector, the method comprising:
studying a sample size of individual specimens of a medical fluid injector type, the injector type comprising a motor drive control and a motor controlled by the motor drive control to inject fluid from a fluid container;
forming a model of usage-related changes in efficiency of the injector type based upon parameters-of-usage;
calibrating a motor drive control of a specific injector of the injector type, to compute injection pressure from electrical energy delivered to the motor;
monitoring the parameters-of-usage of the specific injector; and
recomputing the calibration of the motor drive control of the specific injector based upon the monitored parameters-of-usage utilizing the model formed for the injector type.

8. The method of claim 7, wherein the parameters-of-usage comprise a count of the number of injections completed by the specific injector.

9. The method of claim 7, wherein the parameters-of-usage comprise a count of the number of injections performed by the specific injector.

10. The method of claim 7, wherein the parameters-of-usage comprise a cumulative duration of injections performed by the specific injector.

11. The method of claim 7, wherein the parameters-of-usage comprise one or more pressures reached during medical fluid injections by the specific injector.

12. The method of claim 7, wherein the motor is a rotary electrical motor, and the electrical energy delivered to the motor and measured by the control is an electrical current, the drive motor producing an output torque in approximately proportional relation to the electrical current.

13. The method of claim 7, further comprising:
altering the calibration to reflect an increase, over time, in fluid pressure for a given amount of electrical energy delivered.

14. The injector of claim 7, further comprising:
altering the calibration to reflect an increase, over time, in fluid pressure for a given amount of electrical energy delivered.

15. The method of claim 7, wherein the motor is a rotary electrical motor, and electrical energy delivered to the motor and measured by the motor drive control is an electrical current, the motor producing an output torque in approximately proportional relation to the electrical current.

16. The method of claim 7, wherein the fluid container comprises a syringe.

17. The method of claim 7, wherein the fluid container comprises a fluid-containing bag.

18. The injector of claim 1, wherein the motor is a rotary electrical motor, and electrical energy delivered to the motor and measured by the motor drive control is an electrical current, the motor producing an output torque in approximately proportional relation to the electrical current.

19. The injector of claim 1, wherein the fluid container comprises a syringe.

20. The injector of claim 1, wherein the fluid container comprises a fluid-containing bag.
